(19) United States
(12) Patent Application Publication
(10) Pub. No.: US 2015/0045696 A1
(43) Pub. Date: Feb. 12, 2015

(54) STEERABLE DILATOR

(71) Applicant: Oscor Inc., Palm Harbor, FL (US)

(72) Inventor: Thomas P. Osypka, Palm Harbor, FL (US)

(73) Assignee: OSCOR INC., Palm Harbor, FL (US)

(21) Appl. No.: 14/455,239

(22) Filed: Aug. 8, 2014

Related U.S. Application Data
(60) Provisional application No. 61/863,998, filed on Aug. 9, 2013.
(60) Provisional application No. 61/886,132, filed on Oct. 3, 2013.
(60) Provisional application No. 61/869,140, filed on Aug. 23, 2013.

(51) Int. Cl. A61M 29/02; A61M 5/14; A61M 25/00; A61B 17/12; A61M 25/01; A61M 25/09

(52) U.S. Cl. CPC A61M 29/02; A61M 25/0136; A61M 25/09; A61M 25/0068; A61M 25/0108; A61M 25/002; A61B 17/12013; A61M 5/14; A61M 2025/0177; A61B 2017/00938
USPC 600/585; 606/108; 606/130

(57) ABSTRACT

A steerable dilator includes a dilator body for holding and guiding a sheath, the dilator body having a deflectable distal end portion, and at least one lateral passage configured to accommodate at least one steering cable and a steering handle operatively associated with a proximal end portion of the dilator and having an actuation mechanism operatively connected to the at least one steering cable accommodated within the at least one lateral passages of the dilator for steering the deflectable distal end portion of the dilator in at least one direction.

### CROSS-REFERENCE TO RELATED APPLICATIONS

This application claims the benefit of and priority to U.S. Provisional Application No. 61/863,998, filed Aug. 9, 2013, U.S. Provisional Application No. 61/869,140, filed Aug. 23, 2013, and U.S. Provisional Application No. 61/886,132, filed Oct. 3, 2013, the contents of each being incorporated by reference herein in their entirety.

### BACKGROUND OF THE INVENTION

1. Field of the Invention

The subject invention is directed to steerable medical devices such as steerable dilators for dilators and handles therefor, which are adapted and configured for the introduction and placement of diagnostic and therapeutic devices into the human vasculature.

2. Description of Related Art

There are many instances where physicians must introduce diagnostic and therapeutic devices such as diagnostic and therapeutic electrodes, ultrasound transducers, biopsy devices and other surgical tools into the body. The diagnostic and therapeutic devices are often carried by catheters (a “sheath”) which allow physicians to gain access to the body in a minimally invasive manner by way of bodily lumens. In cardiac treatment, for example, a sheath is advanced through a main vein or artery into the region of the heart that is to be treated.

Guiding sheaths and dilators are also commonly used to introduce balloon catheters and stents into the vascular system (e.g., for percutaneous transvascular coronary angioplasty), to introduce cardiac pacing leads into the coronary sinus (e.g., for left ventricular pacing and cardiac resynchronization procedures), or to introduce radiofrequency ablation catheters into the left atrium (e.g., for treatment of atrial fibrillation) or into the renal artery for renal denervation procedures.

Guiding sheaths and dilators typically come in French sizes ranging from 4 F all the way to 12 F, in some cases even 18 F. Some examples feature an inner lumen extending from the proximal portion all the way to the distal tip section. The inner lumen often has a PTFE liner to make the insertion of a device therethrough as easy and as smooth as possible.

Some situations require the use of a dilator that can be inserted into a sheath to make the sheath more rigid and/or straight for insertion. Presently, no steerable dilator is available and capable of precise movement for guiding a dilator to a location.

There is therefore a need in the art for steerable dilators and handle assemblies therefor which provide relatively precise directional steering and versatility.

### SUMMARY

In at least one aspect of this disclosure, a steerable dilator includes a dilator body for holding and guiding a sheath, the dilator body having a deflectable distal end portion, and at least one lateral passage configured to accommodate at least one steering cable and a steering handle operatively associated with a proximal end portion of the dilator and having an actuation mechanism operatively connected to the at least one steering cable accommodated within the at least one lateral passages of the dilator for steering the deflectable distal end portion of the dilator in at least one direction.

The at least one lateral passage can include two diametrically opposed lateral passages and the at least one steering cable includes two steering cables, one for each lateral passage.

The dilator can include a central lumen defined thereby. A hemostatic seal can be operatively associated with the proximal end portion of the dilator in fluid communication with the central lumen.

The steering handle can be one of mono-direction, bi-directional, or multi-directional. The dilator can have an outer diameter size ranging from about 4 F to about 18 F.

The proximal end portion of the dilator can extend through the steering handle to a proximal end thereof. The dilator can include a single tube of material defining the dilator body.

A sheath can be disposed on the dilator body for placement into vasculature of a patient, the sheath including at least one of an infusion port or a hemostatic seal. A flexible guide wire can be included for introduction through the axial passage of the dilator body.

The dilator can include a tapered distal tip. The dilator can include a hydrophobic coating. In some embodiments, the dilator can include a soft atraumatic tip portion disposed at the distal end portion. The distal end portion of the dilator can include a radiopaque marker band.

In at least one aspect of this disclosure, a kit for placing a surgical device in the vasculature of patient can include an enclosure, a steerable dilator as disclosed herein disposed in the enclosure, and a sheath disposed within the enclosure configured to be disposed on the dilator. The sheath can further include an infusion port. The sheath can further include includes a hemostatic seal on a proximal portion thereof configured to seal about the dilator body or other medical device.

The kit can further include a sheath disposed within the enclosure configured to be disposed on the dilator. The kit can further include a guide wire disposed within the enclosure.

### BRIEF DESCRIPTION OF THE DRAWINGS

So that those skilled in the art to which the subject invention appertains will readily understand how to make and use the subject invention without undue experimentation, reference may be had to the figures, wherein:

FIG. 1A is an illustration of an embodiment of a bi-directional steerable dilator in accordance with this disclosure, shown with a sheath disposed thereon for insertion into the human vasculature, e.g., intracardiac, renal, and/or transseptal placement;

FIG. 1B is an enlarged illustration of a lockable actuation mechanism disposed on the handle portion of the dilator of FIG. 1A;

FIG. 1C is an illustration of a section of the dilator of FIG. 1A, showing the outer surface of the dilator including a hydrophobic coating;

FIG. 1D is an illustration of a cross-section of the dilator of FIG. 1A, showing a steering wires disposed in an interior shaft portion of the dilator;

FIG. 1E is an illustration of an embodiment of a tip portion of the dilator of FIG. 1A, shown including a central lumen;

FIG. 1F is an illustration of an embodiment of a kit containing the steerable dilator of FIG. 1A, a sheath, and a guide wire;

FIG. 1G is a top plan view of the steerable dilator of FIG. 1A;

FIG. 1H is a bottom plan view of the steerable dilator of FIG. 1A;

FIG. 1I is a side elevational view of the dilator of FIG. 1A, with the steering wires shown;

FIG. 1J is a cross-sectional view of the dilator shown in FIG. 1I taken along line 1J-1J;

FIG. 1K is a cross-sectional view of the dilator shown in FIG. 1I taken along line 1K-1K;

FIG. 1L is a cross-sectional view of the handle portion of the steerable dilator, with reference to FIG. 1H;

FIG. 1M is a side, elevational view of the dilator of FIG. 1A, shown with an embodiment of an over molded hub that supports a hemostatic seal;

FIG. 1N is a longitudinal cross-sectional view of the dilator of FIG. 1M, illustrating the various internal structures and steering wires;

FIG. 2A is a side elevational view of the handle of the steerable dilator of FIG. 1A;

FIG. 2B is a perspective exploded view of a first half of the handle of FIG. 2A showing an actuation mechanism and other internal components relative to a first half of the handle housing;

FIG. 2C is a perspective exploded view of a second half of the handle of FIG. 2A showing an actuation mechanism and other internal components relative to a second half of the handle housing;

FIG. 2D is a front view of the actuation mechanism of FIGS. 2B and 2C;

FIG. 2E is a rear view of the actuation mechanism of FIGS. 2B and 2C;

FIG. 2F is a side view of the actuation mechanism of FIGS. 2B and 2C;

FIG. 2G is a bottom plan view of a locking tab of the locking mechanism of the device of FIG. 2A;

FIG. 2H is a cross-sectional side view of a locking tab of the locking mechanism of the device of FIG. 2A;

FIG. 2I is a perspective view of an embodiment of the friction lock member of FIGS. 2B and 2C, showing camming surfaces and locking divots on the camming surfaces;

FIG. 2J is a top plan view of an embodiment of the friction lock member of FIG. 2I;

FIG. 2K is a side view of an embodiment of the friction lock member of FIG. 2I;

FIG. 2L is a cross-sectional side view of an embodiment of the friction lock member of FIG. 2I;

FIG. 2M is a perspective view of a dilator stabilizing member, showing a dilator hole and flange members extending therefrom;

FIG. 2N is a top plan view of the dilator stabilizing member of FIG. 2M;

FIG. 3A is a depiction of the bi-directional steerable dilator of FIG. 1A shown having the distal end portion of the dilator body in a first deflected position;

FIG. 3B is a depiction of the bi-directional steerable dilator of FIG. 3A shown having the distal end portion of the dilator body in a second deflected position;

FIG. 3C is a depiction of the bi-directional steerable dilator of FIG. 3A shown having the distal end portion of the dilator body having a sheath disposed thereon and in a second deflected position;

FIG. 4A is an illustration of an embodiment of a bi-directional steerable dilator having another embodiment of a handle assembly in accordance with this present disclosure;

FIGS. 4B-4D illustrate aspects of the handle assembly of the steerable dilator of FIG. 4A;

FIG. 4E is an enlarged partial cross-sectional view of the handle assembly shown in

FIG. 4D, illustrating the internal components of the actuation assembly that activates the two steering wires which control the bi-directional movement of the distal end portion of the dilator; and

FIG. 4F is a cross-sectional view of the dilator of FIG. 4A, illustrating the central lumen and opposed passages that accommodate the two steering wires; and

FIG. 5 is an in-situ view of an embodiment of this disclosure disposed within a renal artery during a medical procedure.

### ENABLING DESCRIPTION OF THE INVENTION

Referring now to the drawings wherein like reference numerals identify similar structural features or elements of the disclosed devices, embodiments of this disclosure are directed a steerable dilator assembly 110 that is adapted and configured to introduce a sheath into the vascular system of a patient during an endovascular surgical procedure.

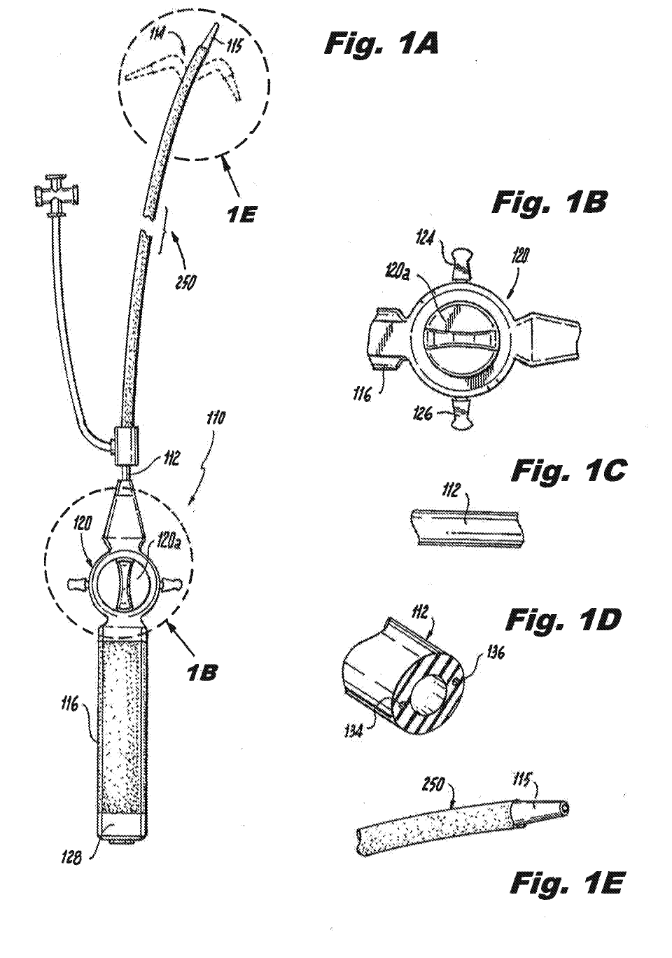

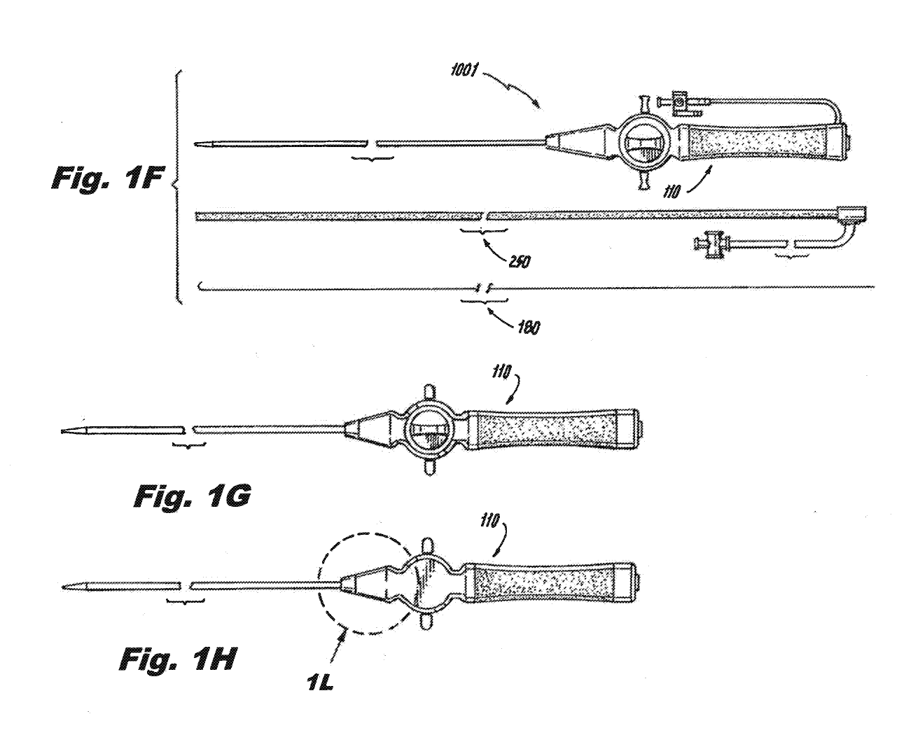

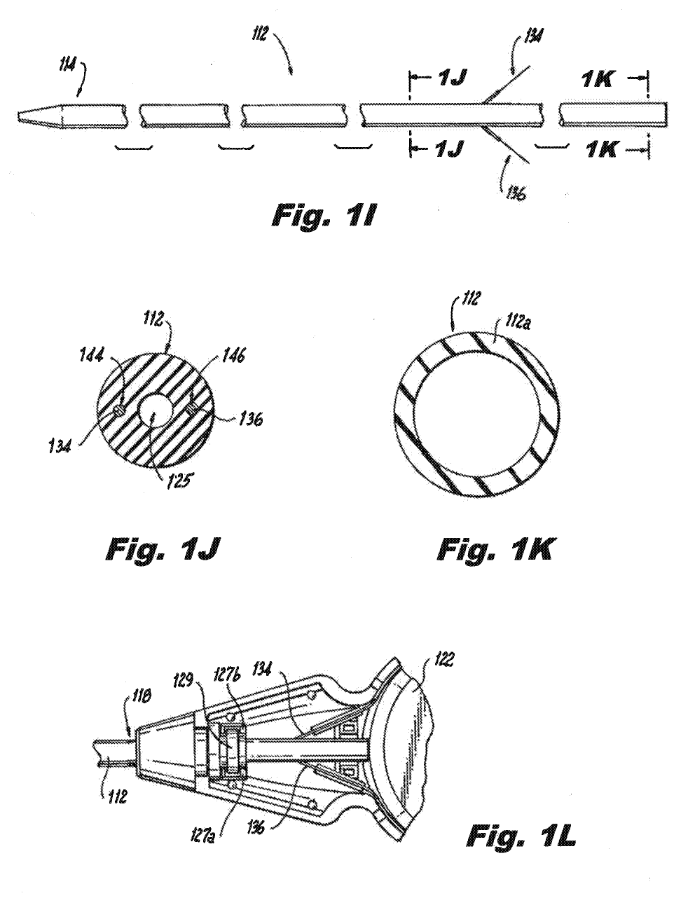

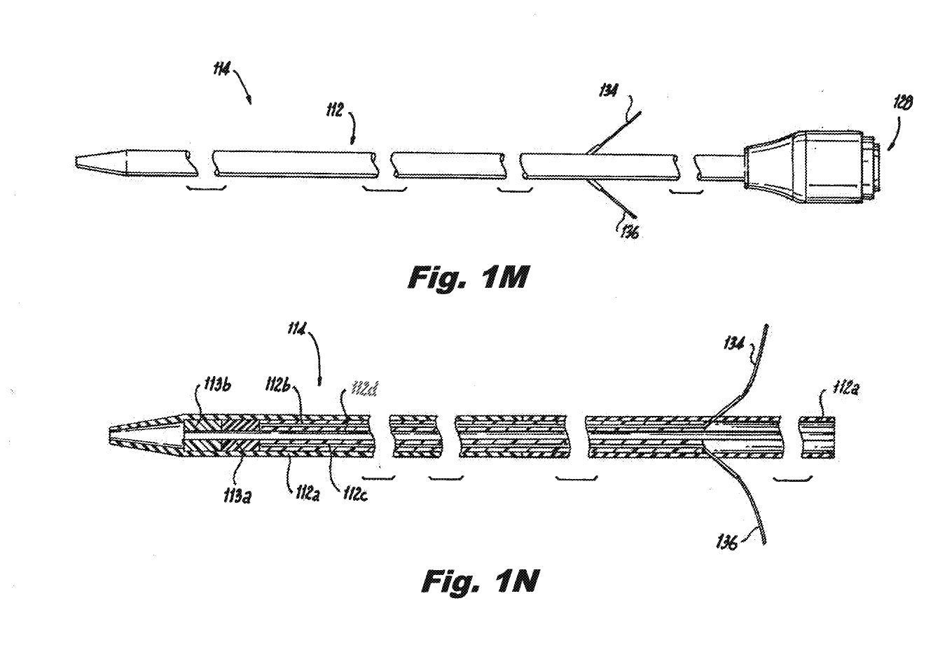

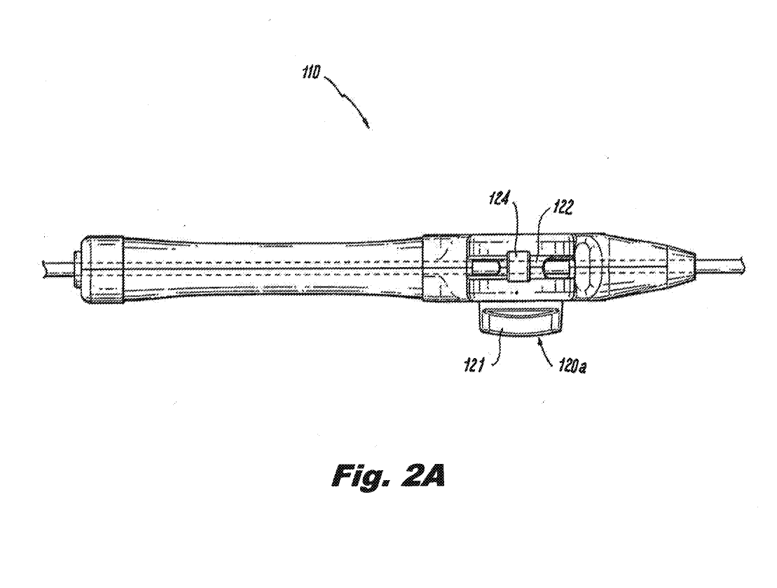

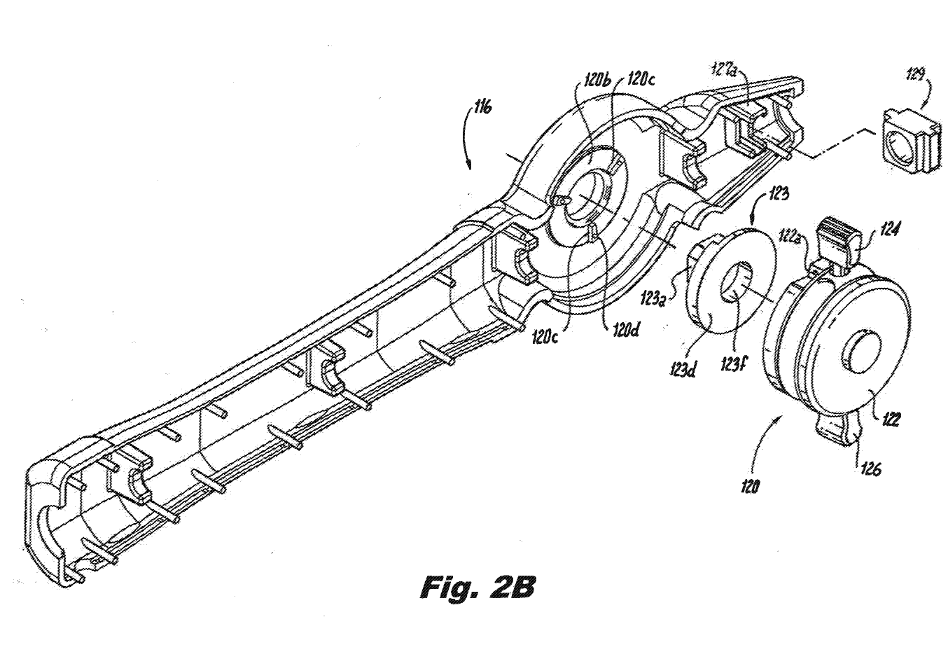

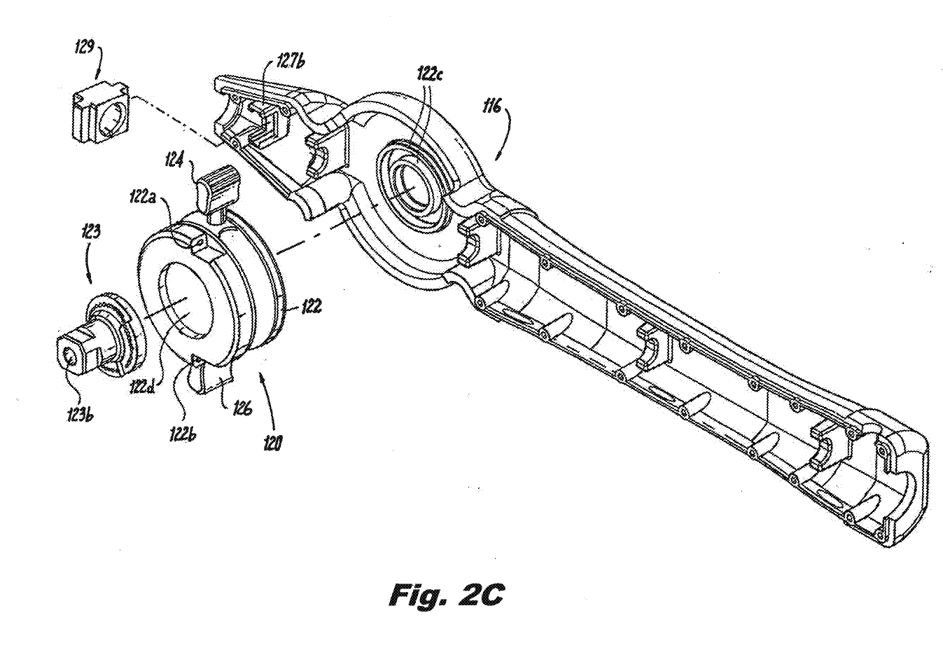

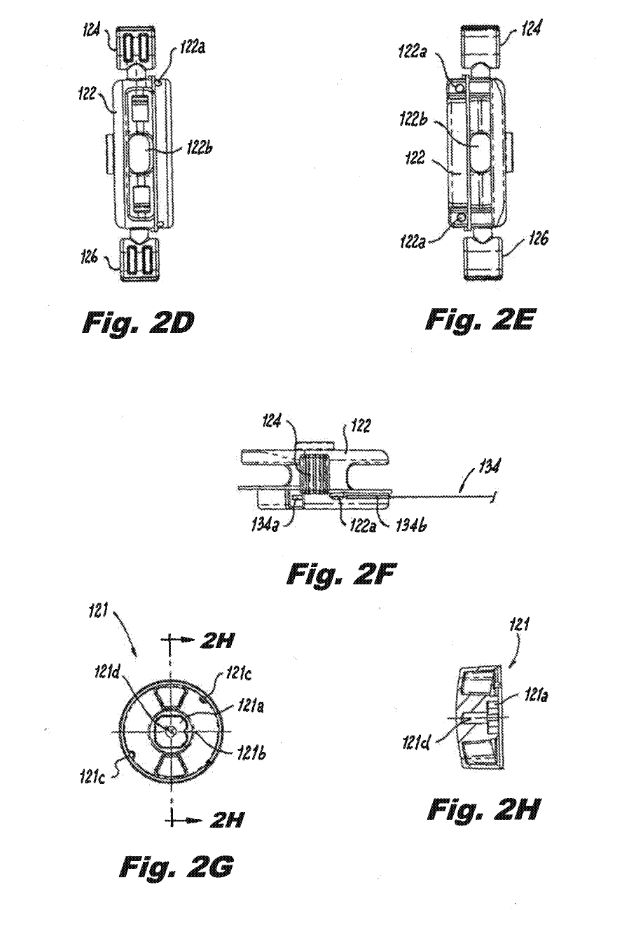

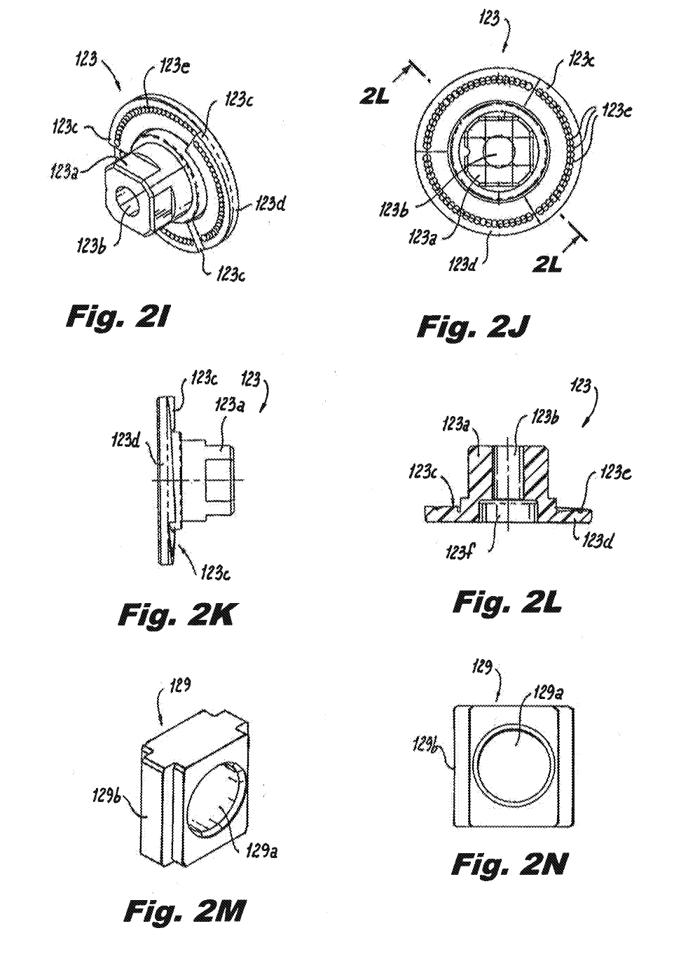

In at least one aspect of this disclosure, referring generally to FIGS. 1A-2N, the steerable dilator 110 can include an elongated dilator body 112 having a deflectable distal end portion 114. The dilator body 112 can include and/or define a central lumen 125 and a pair of diametrically opposed lateral passages 144, 146 to accommodate a corresponding pair of steering cables 134, 136. While the embodiment of FIG. 1A is shown as having two lateral passages 144, 146 and two steering cables 134, 136, any suitable number of passages and/or cables are contemplated herein, e.g., one, two, three, four, or more. The central lumen 125 of the dilator body 112 can extend through the dilator body 112 for accommodating a guide wire. A guide wire can enable the dilator body 112 to more readily traverse the vascular system of a patient during a surgical procedure.

The dilator body 112 can have any suitable outer diameter size for a desired use. In some embodiments, the outer diameter size of the dilator body 112 ranges from about 4 F to about 18 F. In some embodiments, the outer diameter of the dilator body 112 is about 5 F. The deflectable distal end portion 114 of the dilator body 112 can include a tapered distal tip 115 to ease the percutaneous introduction of the dilator body 112 into the vascular system of a patient during a surgical procedure.

In some embodiments, the proximal end portion 118 of the dilator body 112 can extend through the steering handle 116 to a proximal end thereof. A hemostatic seal 128 can also be operatively associated with the proximal end portion 118 of the dilator body 112 such that the hemostatic seal 128 is in fluid communication with the central lumen 125 thereof. In at least some embodiments, the hemostatic seal 128 can provide an effective seal for a guide wire of about 0.014 inches. It is contemplated that a seal 128 and or central lumen 125 not be included in some embodiments.

The dilator body 112 can have a hydrophobic coating or any other suitable and/or desired coating. In some embodiments, the dilator body 112 can be formed from a hydrophobic material.

The dilator body 112 can include a single layer structure at one or more portions thereof. Referring to FIGS. 1D and 1J, a portion of the dilator body 112 can include a tube of any suitable material (e.g., biocompatible plastic, fabric, metal) having steering wires 134, 136 disposed therein. Any other suitable layer structure and/or reinforcement is contemplated herein. It is also contemplated that the dilator body 112 be a solid piece of material without a central lumen therein.

The thickness of the dilator body 112 can be modified at sections thereof to allow the flexibility of dilator body 112 to be controlled. In some embodiments, the flexibility of the dilator body 112 can be modified as a function of length of the dilator body 112 to control the point along the dilator body 112 that the distal portion deflects about and/or degrees of deflection of portions of the dilator body 112.

As shown in FIG. 1O, such a portion of the dilator body 112 (e.g., a proximal portion within the handle) can include a thinner layer and/or a layer of a differing material or thickness from the distal portion thereof.

Referring specifically to FIG. 1N, the dilator body 112 can also include a radiopaque marker 113b. The radiopaque marker 113b can be any suitable shape (e.g., a cylinder) and can include any radiopaque material and/or the like for locating the radiopaque marker 113b in situ to enable the visual guidance of the dilator 110 through the vascular system of a patient using a suitable imaging system.

The distal end portion 114 can also include and anchor member 113a disposed therein configured to anchor the steering wires 134, 136 to the distal end portion 114. The anchor member 113a can be of any suitable shape (e.g., cylindrical) and mounted within the distal end portion 114 of the dilator body 112 such that the anchor member 113a does not move relative to the distal tip when pulled on by the steering wires 134, 136.

Referring to FIGS. 1A and 1F, the steerable dilator 110 can further include and/or be operative with a sheath 250 dimensioned to be placed around the dilator body 112 (shown in a kit 1001). The sheath 250 can include an interior lumen extending therethrough (not shown) for accommodating the dilator body 112. As shown, the sheath 250 may be dimensioned to not be as long as the dilator body 112. That is, the distal end of the sheath 250 can be made to not extend entirely over the distal end portion 114 of the dilator body 112, but any suitable length is contemplated herein.

It is also envisioned that the sheath 250 can have an outer diameter size ranging from about 4 F to about 12 F, and in some instances as large as about 18 F. Any other suitable size is contemplated herein such that the sheath 250 can fit around one or more embodiments of the dilator body 412. Furthermore, the sheath 250 can be relatively compliant so that it is readily deflectable together with the distal end portion 114 of the dilator body 112.

A valve housing 252 can be operatively associated with a proximal end portion of the sheath 250. The valve housing 252 includes a hemostatic valve that sealingly isolates an interior lumen of the sheath 250 when the dilator body 112 is accommodated therein.

The valve housing 252 can also include an infusion port 254 (e.g., including a conventional leur fitting) for aspirating and flushing the interior lumen of the sheath 250. The valve housing 252 and the associated infusion port 254 can be excluded in certain embodiments.

It is envisioned that the sheath 250 can be configured as a peal-away sheath that is formed with at least one score line. In such an embodiment, after the placement of a medical device (e.g. a cardiac lead) in the vascular system of a patient, the sheath 250 can be readily removed from the surgical site by pealing the sheath 250 away or otherwise removing it.

The steerable dilator 110 can further include a steering handle 116 operatively associated with a proximal end portion 118 of the dilator body 112 and an actuation mechanism 120 that is operatively connected to the pair of steering cables 134, 136 accommodated within the opposed lateral passages 144, 146 of the dilator body 112 for steering the deflectable distal end portion 114 of the dilator body 112 in one or more directions (e.g., bi-directionally as shown in this embodiment).

Referring to FIG. 2A-2C, a first half 116a of the handle 116 includes lock surface 120b on the inside portion of the first half 116a defining an opening between the inside of the handle 116 and the outside thereof. The lock surface 120b includes one or more cam protrusions 120c extending from the lock surface 120b. The cam protrusions 120c include one or more lock protrusion 120d. The cam protrusions 120c are configured to engage a friction lock member 123 described in more detail below.

As shown in FIGS. 2B and 2C, the first and second halves 116a, 116b are dimensioned to accept the actuation mechanism 120 therein. As shown in FIG. 2C, the second half 116b can include ridges 122c or any other surface inside the second half 116b to allow the central hub 122 of the actuation mechanism 120 to rotate relative to the handle 116.

Referring to FIGS. 2B, 2C, and 2D-2F, the actuation mechanism 120 of the steering handle 116 can include a central hub 122 connected to the actuators 124, 126. The central hub 122 can define a passageway 122b configured to allow the dilator body 112 to pass therethrough. The passageway 122b is dimensioned to prevent bending or moving the portion of the dilator body 112 passing therethrough between the limits of actuation of the actuation mechanism 120.

The flexible steering cables 134, 136 can be secured to the periphery of the central hub 122 of actuation mechanism 120. For example, the central hub 122 can define wire holes 122a which steering cables 134, 136 can pass through. The steering cables 134, 136 can be secured to the central hub 122 using a crimp 134a or any other suitable attachment. In some embodiments, a guide member 134b can be disposed around the steering cable 134, 136 distal of the wire holes 122a to prevent the steering cables from bending around the central hub 122 allowing the steering cables 134, 136 to angle inwardly toward the dilator body 112 without bending the cables 134, 136.

Also as shown best in FIG. 2C, the central hub 122 can define a friction lock cavity 122d configured to accept a friction lock member 123 therein. As shown the actuation mechanism 120 can be a single molded piece of material (e.g., suitable plastic), but any suitable combination of parts is contemplated herein.

As shown in FIG. 2B, a friction lock member 123 is configured to be disposed between the actuation mechanism 120 and the first half 116a of the housing 116. Referring to FIG. 2I-2L, the friction lock member 123 can include a pedestal portion 123a defining a hole 123b therethrough and a flange portion 123d extending from the pedestal portion 123a. The flange portion 123d can define a frictional surface for engaging the central hub 122 of the actuation mechanism 120. In addition, the flange portion 123d includes one or more camming surfaces 123c which can define locking divots 123e. The camming surfaces 123c can include any suitable shape (e.g., ramped as shown).

Referring to FIGS. 2G and 2H, a locking tab 121 of the locking mechanism 120a can include a body 121b shaped to be gripped by a user and a pedestal cavity 121a defined therein dimensioned to receive the pedestal portion 123a of the friction lock member 123. An attachment hole 121d can be included within the pedestal cavity 121a to allow a screw or other suitable member to affix thereto to attach the friction lock member 123 to the locking tab 121.

Referring additionally to FIG. 2L, an attachment member (e.g., a screw) can be passed through hole 123b and into attachment hole 121d to attach the friction lock member 123 to the locking tab 121 in a sandwich with the housing 116 therebetween. The attachment member can be dimensioned such that a head of the attachment member can seat into head cavity 123f of the and an attachment portion of the attachment member can advance into attachment hole 121d sufficiently to sufficiently sandwich the housing 116 between the locking tab 121 and the friction lock member 123 against the lock surface 120b while still allowing the assembly to rotate when the locking tab 121 is rotated.

In this regard, the cam protrusions 120c maintain contact with the camming surfaces 123c such that when the locking tab 121 is rotated, the friction lock member 123 rotates therewith causing the relative position of the cam protrusions 120c to change relative to the camming surfaces 123c. When the cam protrusions 120c are in contact with a thicker portion of the camming surfaces 123c, the friction lock mechanism 123 is moved closer to the central hub 122, causing the friction surface of the flange 123d to push upon the central hub 122 to produce more frictional resistance to rotation of the hub 122. The lock protrusions 120d mate with the locking divots 123e to prevent the locking member 123 from slipping back down the cam path and provide a tactile feedback while turning the locking tab 120a between an unlocked position and a locked position.

Any other suitable locking mechanism 120a and/or components thereof to prevent or inhibit movement of the actuators 124, 126 is contemplated herein.

Referring to FIGS. 2M and 2N in conjunction with FIGS. 2B and 2C, a dilator stabilizing member 129 can include a dilator hole 129a dimensioned for the dilator to pass therethrough and flange members 129b extending therefrom. The dilator stabilizing member 129 is configured to fit within stabilizing member holders 127a, 127b that are disposed on the inside of the first and second halves 116a, 116b, respectively. The dilator stabilizing member 129 allows the dilator body 112 to be directed at the distal end of the handle 116 so that motion of the dilator body 112 within the handle 116 can be resisted.

When assembled and in an unlocked position, the actuation mechanism 120 can rotate between first half 116a and second half 116b of the housing 116 to steer the distal tip of the dilator body 112. The locking tab 121 can be moved between an unlocked position such that the actuation mechanism 120 can rotate without substantial resistance and a locked position such that a resistance to rotation is created by the locking mechanism 120a.

Additionally, positions between the unlocked and locked position can be selected by a user such that the sensitivity of control of the distal end of the dilator body 112 is modified. In such an instance, the amount of force provided by the locking mechanism 120a can be modified by turning the locking mechanism 120a to a particular position between the locked position and the unlocked position, thereby altering the force required to deflect the distal end portion 114. This can be used to allow the user to modify the sensitivity of the actuating mechanism 120 using the locking mechanism 120a.

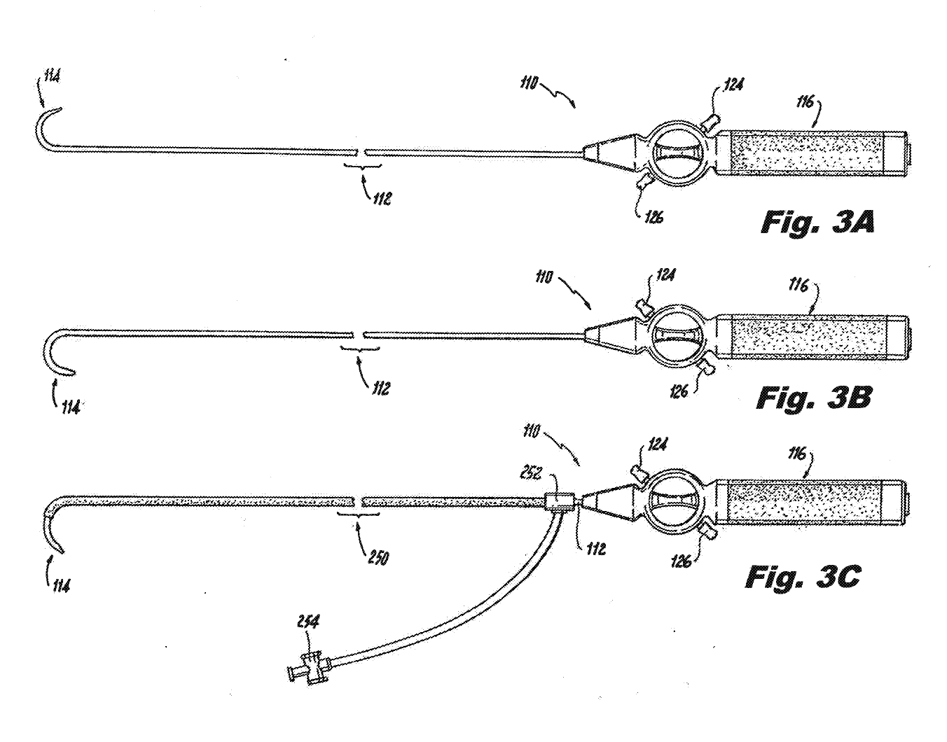

Referring to FIGS. 3A-3C the dilator body 112 is shown being steered in a first deflected position (FIG. 3A) to a second deflected position (FIG. 3B). Also shown is a sheath 250 disposed in the dilator 110 in the second steered position (FIG. 3C). Once introduced into the vascular system of a patient, the sheath 250 can be subsequently used, for example, to introduce balloon catheters and stents into the vascular system, to introduce cardiac pacing leads into the coronary sinus, or to introduce radiofrequency ablation catheters into the left atrium for treatment of atrial fibrillation or the renal artery for renal denervation procedures and/or other medical procedures.

In use, manipulation of the actuators 124 and 126 in clockwise and counter-clockwise directions causes the corresponding movement of the central hub and steering cables 134 and 136. This results in the bi-directional deflection of the distal end portion 114 of the dilator body 112. It is contemplated that clockwise actuator motion can lead to a counter-clockwise tip deflection, and vice versa. The actuation mechanism 120 controls the orientation of the distal end portion of the dilator and can be designed to have any suitable maneuverability (e.g., 180° dual deflection maneuverability).

Referring to FIG. 1F, a kit 1001 for placing a surgical device in the vasculature of patient can include an enclosure (not shown), a steerable dilator 110 as describe herein disposed within the enclosure, a sheath 250 as described above disposed within the enclosure, and a guide wire 180 disposed in the enclosure.

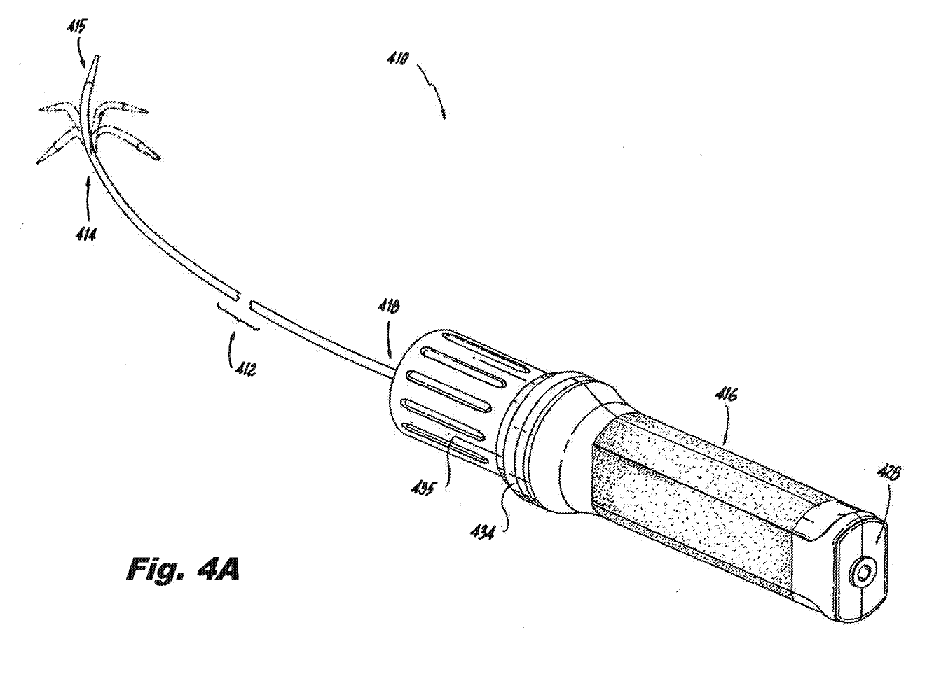

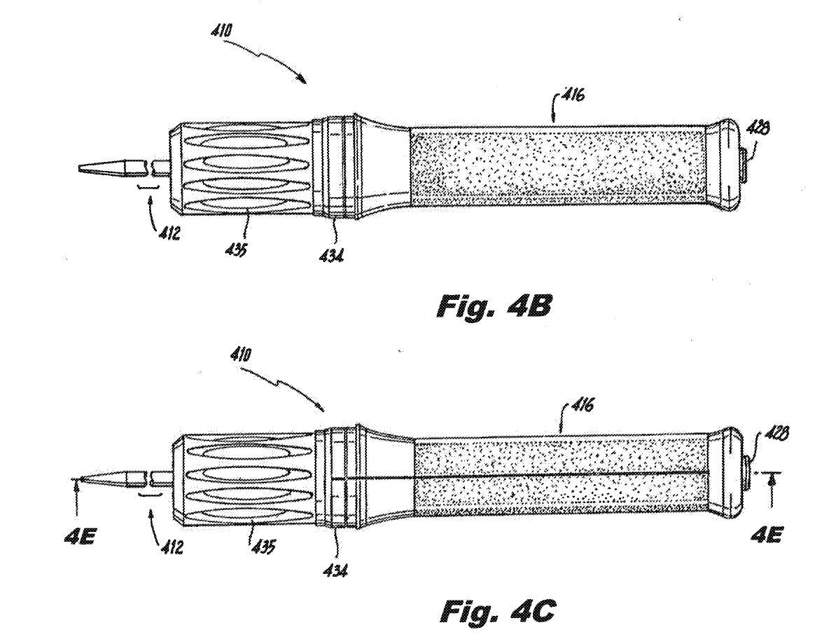

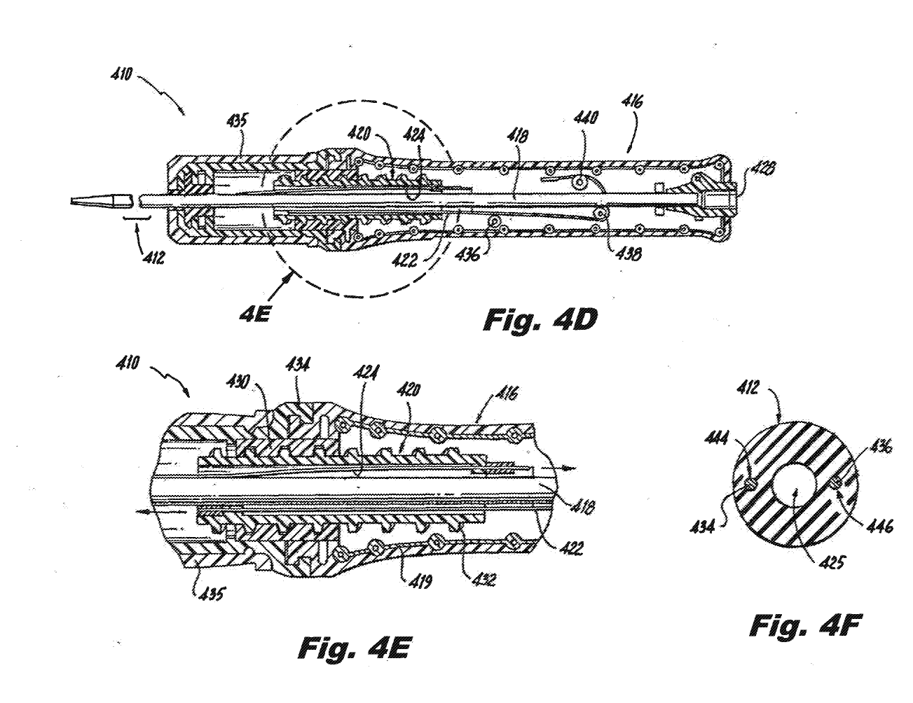

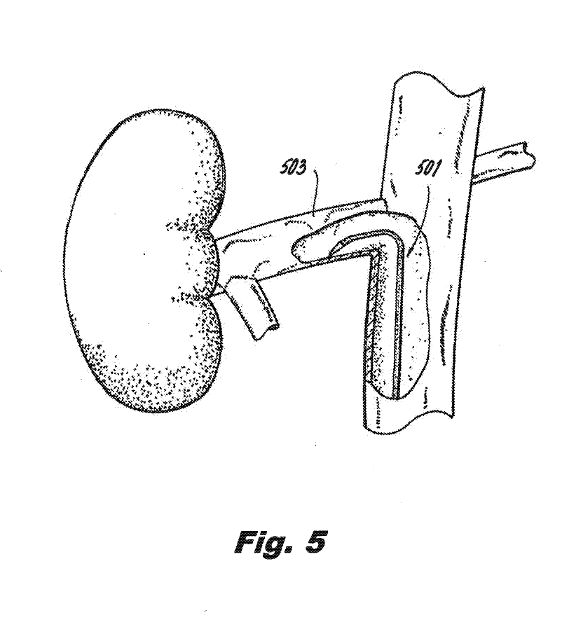

In at least one aspect of this disclosure, referring now to FIGS. 4A-4F, the steerable dilator 410 can include a differing handle assembly 416 than the above described embodiments. The steerable dilator 410 includes an elongated dilator body 412 having a deflectable distal end portion 414 and a central lumen 425 (see FIG. 4F). As shown in FIG. 4A, the distal end portion 414 of dilator body 412 can be adapted and configured to achieve about a 180 degree deflection (e.g., mono-directional, bidirectional). Other suitable maximum deflections are contemplated herein.

Similar to the other dilators described herein, the dilator body 412 can have an outer diameter size ranging from about 4 F to about 18 F. Any other suitable size is contemplated herein. The dilator body 412 is configured to be operable with a sheath 250 as described above in a manner similar to that as described above.

The steerable dilator 410 includes an elongated handle assembly 416 operatively associated with a proximal end portion 418 of the dilator body 412. The proximal end portion 418 of the dilator body 412 can extend through the steering handle 416 to a proximal end thereof.

A hemostatic seal 428 can be operatively associated with the proximal end portion 418 of the dilator body 412 and in fluid communication with the central lumen 425. As disclosed above, a hemostatic seal 428 permits sealed introduction of a guide wire or other suitable medical device.

The dilator body 412 can include a hydrophobic coating and/or a soft atraumatic tip portion 415 similar to those as described above. The tip portion 415 of the dilator body 412 can include a radiopaque marker band similar to marker band 113b as described above.

The handle assembly 416 of steerable dilator 410 includes a body 419 that houses a manually operable actuation mechanism 420. The actuation mechanism 420 can be operatively connected to one or more steering wires 422 and 424. As best seen in FIG. 4F, the steering wires 422 and 424 can be accommodated within opposed lateral passages 426 and 427 of the dilator body 412. As shown in this embodiment, the steering wires 422 and 424 are arranged to control the deflection of the distal end portion 414 of the dilator body 412 in two directions, as described in more detail herein below.

As best seen in FIG. 4E, the actuation mechanism 420 can include a drive nut 430 that is threadably coupled to a worm coil 432. Rotation of the drive nut 430 causes axial translation of the worm coil 432 within the body 419 of the handle assembly 416.

The drive nut 430 and worm coil 432 can include a common thread pitch that is selected to achieve a precise amount of control over the deflection achieved at the distal end portion 414 of the dilator body 412. For example, differing thread pitches advance the worm coil 432 at different rates, allowing more or less motion of the tip relative to the amount of motion of the user, thereby modifying precision. It would be appreciated by those having skill in the art that the more control a surgeon has over the deflection of the distal end of the dilator, the easier it is for that surgeon to accurately steer the dilator body 412 though the vasculature of a patient to the site of a procedure.

The actuation mechanism 420 further includes a manually rotatable torque ring 434 that is operatively connected to the drive nut 430 and configured to be rotated by a user. The torque ring 434 can be positioned adjacent a stationary torque grip 435, thereby enabling a user to maintain a firm grip on the device 410 while rotating the torque ring 434 to achieve the directional deflection of the distal end portion 414 of the dilator body 412.

As shown, the steering wire 422 can be operatively connected or otherwise crimped to a distal end portion of the worm coil 432 of actuation mechanism 420. Also as shown, the other steering wire 424 can be operatively connected or otherwise crimped to a proximal end portion of the worm coil 432. As best seen in FIG. 4D, steering wire 422 can be longer than the steering wire 424.

The longer steering wire 422 can be operatively supported by a pair of guide rollers 436 and 438. Guide roller 436 can be disposed in a stationary position within the body 419 of handle assembly 416. In contrast, guide roller 438 can be dynamically positioned within the body 419 of handle assembly 416, such that the guide roller 438 is operatively associated with a spring biased tension arm 440 that is pivotally mounted within the body 419 of handle assembly 416. As shown in FIG. 4D, the steering wire 422 can be looped around the dynamic guide roller 438 so that it doubles back around toward the crimped end of the wire and then out to the distal end portion 414 of the dilator body 412.

In operation, when the worm coil 432 translates in a distal direction through rotation of drive nut 430, the end of the longer steering wire 422 that is crimped to the distal end portion of the worm coil 432 is pulled in a distal direction. Consequently, the portion of steering wire 422 that double backs around guide roller 438 is pulled in a proximal direction. This causes controlled deflection of the distal end portion 414 of the dilator body 412.

When the worm coil 432 translates in a proximal direction through the reverse rotation of drive nut 430, the shorter steering wire 424 that is crimped to the proximal end portion of worm coil 432 is pulled in a proximal direction therewith. This causes controlled deflection of the distal end portion 414 of dilator body 412 in an opposite direction. At the same time, the crimped end of the longer steering wire 422 moves proximally with the worm coil 432, and the slack in that wire is accommodated by the spring biased tension arm 440.

The actuation mechanism 420 and the arrangement of steering wires 422, 424 allows for the bidirectional deflection of the distal end portion 414 of the dilator body 412 using a worm coil 432 that has a single uniform thread pitch. Those skilled in the art will readily appreciate that the amount or degree of deflection, and the associated precision steering that can be achieved, can be adjusted by changing the thread pitch of the drive nut 430 and worm coil 432 as described above. That is, a greater amount of precision for the deflection of the distal end portion 414 of dilator body 412 can be achieved by increasing the thread pitch of the drive nut 430 and worm coil 432.

The devices, methods, and systems of the present disclosure, as described above and shown in the drawings, provide for steerable medical devices with superior properties including advanced directional and precision control. While the apparatus and methods of the subject disclosure have been shown and described with reference to embodiments, those skilled in the art will readily appreciate that changes and/or modifications may be made thereto without departing from the spirit and scope of the subject disclosure.

What is claimed is:

1. A steerable dilator, comprising:
a) a dilator body for holding and guiding a sheath, the dilator body having a deflectable distal end portion, and at least one lateral passage configured to accommodate at least one steering cable; and
b) a steering handle operatively associated with a proximal end portion of the dilator and having an actuation mechanism operatively connected to the at least one steering cable accommodated within the at least one lateral passages of the dilator for steering the deflectable distal end portion of the dilator in at least one direction.

2. The steerable dilator of claim 1, wherein the at least one lateral passage includes two diametrically opposed lateral passages and the at least one steering cable includes two steering cables, one for each lateral passage.

3. The steerable dilator of claim 2, wherein the dilator includes a central lumen defined thereby.

4. The steerable dilator of claim 3, wherein a hemostatic seal is operatively associated with the proximal end portion of the dilator in fluid communication with the central lumen.

5. The steerable dilator of claim 3, wherein the steering handle is one of mono-direction, bi-directional, or multi-directional.

6. The steerable dilator of claim 5, wherein the dilator has an outer diameter size ranging from about 4 F to about 18 F.

7. The steerable dilator of claim 5, wherein the proximal end portion of the dilator extends through the steering handle to a proximal end thereof.

8. The steerable dilator of claim 5, wherein the dilator includes a single tube of material defining the dilator body.

9. The steerable dilator of claim 5, further comprising a sheath disposed on the dilator body for placement into vasculature of a patient, the sheath including at least one of an infusion port or a hemostatic seal.

10. The steerable dilator of claim 9, further comprising a flexible guide wire for introduction through the axial passage of the dilator body.

11. The steerable dilator of claim 1, wherein the dilator includes a tapered distal tip.

12. The steerable dilator of claim 1, wherein the dilator includes a hydrophobic coating.

13. The steerable dilator of claim 1, wherein the dilator includes a soft atraumatic tip portion disposed at the distal end portion.

14. The steerable dilator of claim 1, wherein the distal end portion of the dilator includes a radiopaque marker band.

15. A kit for placing a surgical device in the vasculature of patient, comprising:
a) an enclosure; and
b) a steerable dilator disposed within the enclosure, wherein the steerable dilator includes:
i) a dilator body having a deflectable distal end portion, and at least one lateral passage configured to accommodate at least one steering cable;
ii) a steering handle operatively associated with a proximal end portion of the dilator and having an actuation mechanism operatively connected to the at least one steering cable accommodated within the at least one lateral passages of the dilator for steering the deflectable distal end portion of the dilator in at least one direction; and
iii) a sheath disposed within the enclosure configured to be disposed on the dilator.

16. The kit of claim 15, wherein the sheath further includes an infusion port.

17. The kit of claim 15, wherein the sheath further includes a hemostatic seal on a proximal portion thereof configured to seal about the dilator body or other medical device.

18. The kit of claim 15, further comprising a guide wire disposed within the enclosure.
